(19) United States
(12) Patent Application Publication
(10) Pub. No.: US 2009/0012296 A1
(43) Pub. Date: Jan. 8, 2009

(54) Processes for the preparation of crystalline form beta of imatinib mesylate

(76) Inventor: Alexandr Jegorov, Dobra Voda (CZ)

Correspondence Address: KENYON & KENYON LLP, ONE BROADWAY, NEW YORK, NY, 10004, US

(21) Appl. No.: 12/156,154

(22) Filed: May 29, 2008

Related U.S. Application Data
(60) Provisional application No. 60/932,244, filed on May 29, 2007.

(51) Int. Cl. C07D 403/14

(52) U.S. Cl. 544/295

(57) ABSTRACT

Provided is a process for the preparation of crystalline form β of Imatinib mesylate.

### CROSS REFERENCE TO RELATED APPLICATIONS

The present application claims the benefit of the following U.S. Provisional Patent Application No. 60/932,244, filed May 29, 2007. The contents of this application is incorporated herein by reference.

### FIELD OF THE INVENTION

The present invention encompasses processes for the preparation of β of imatinib mesylate.

### BACKGROUND OF THE INVENTION

Imatinib mesylate, 4-(4-methylpiperazin-1-ylmethyl)-N-[4-methyl-3-[(4-pyrinin-3-yl)pyrimidin-2-yloamino]phenyl]benzamide mesylate, a compound having the chemical structure,

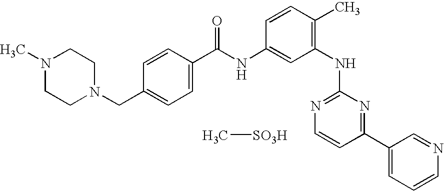

is a protein-tyrosine kinase inhibitor, especially useful in the treatment of various types of cancer and can also be used for the treatment of atherosclerosis, thrombosis, restenosis, or fibrosis. Thus imatinib can also be used for the treatment of non-malignant diseases. Imatinib is usually administered orally in the form of a suitable salt, e.g., in the form of imatinib mesylate.

International Patent Application Nos. WO 99/03854, WO 2005/077933, WO 2005/095379, WO 2004/106326, WO 2006/054314, WO 2006/024863 (also published as US 2007/265288), WO 2006/048890, US2006/0030568, WO 2007/023182, WO 2007/059963, and U.S. Pat. No. 6,894,051 apparently describe crystalline forms of imatinib mesylate designated Forms H1, α, α2, β, δ, ε, I, II, F, G, H, I, K, and amorphous imatinib mesylate.

WO 99/03854, US2006/0030568 and U.S. Pat. No. 6,894,051 discloses form β, which is said to be characterized by a PXRD pattern having peaks at 9.7, 13.9, 14.7, 17.5, 18.2, 20.0, 20.6, 21.1, 22.1, 22.7, 23.8, 29.8 and 30.8±0.2 degrees two theta.

Additional crystalline forms of imatinib mesylate are described in WO 2007/136510.

U.S. Pat. No. 6,894,051 also discloses a process for preparing form β by “digesting another crystal form, especially the α-crystal form, or an amorphous starting material of the methanesulfonic acid addition salt of imatinib with a suitable polar solvent, especially an alcohol, most especially methanol, or also a ketone (especially in a mixture with water, for example water/acetone), typically acetone, a N,N-di-lower alkyl-lower alkanecarboxamide, typically N,N-dimethylformamide or -acetamide, or a hydrophilic ether, typically dioxane, preferably in the presence of some water, or mixtures thereof, in suspension at a suitable temperature, preferably a temperature between 20 and 50° C., for example at about 25° C.”.

The above patent discloses an additional process for preparing form β “dissolving another crystal form, especially the α-crystal form, or an amorphous starting material of the methanesulfonic acid addition salt of imatinib with a suitable polar solvent, such as especially an alcohol, typically methanol or ethanol, a ketone (especially in a mixture with water, for example water/acetone), typically acetone, a N,N-di-lower alkyl-lower alkanecarboxamide, typically N,N-dimethylformamide or -acetamide, or a hydrophilic ether, typically dioxane, or mixtures thereof, preferably in the presence of some water, at a suitable temperature, especially after heating the solvent, or while warming during the dissolution process, in both cases preferably to 25° C. up to the reflux temperature of the reaction mixture, and than initiating crystallization by adding a small amount of the β-crystal form as seeds crystal at a suitable temperature, for example between 0 and 70° C., preferably between 20 and 70° C.”.

Another process for preparation of form β is described in WO 2007/023182, wherein form δ is converted to form β by a similar process.

The present invention affords a new process for preparing form β.

### SUMMARY OF THE INVENTION

In one embodiment, the present invention provides a process for preparing crystalline form β comprising suspending crystalline Imatinib mesylate form IV in a solvent selected from the group consisting of: a ketone, a cyclic ether, an ester, or mixtures thereof.

### BRIEF DESCRIPTION OF THE FIGURES

FIG. 1 illustrates a powder X-ray diffraction pattern for imatinib mesylate Form IV.

FIG. 2 illustrates a solid-state ¹³C NMR spectrum of imatinib mesylate Form IV in the 100-180 ppm range.

FIG. 3 illustrates a solid-state ¹³C NMR spectrum of imatinib mesylate Form IV.

FIG. 4 illustrates a powder X-ray diffraction pattern for imatinib mesylate Form beta.

FIG. 5 illustrates a powder X-ray diffraction pattern for imatinib mesylate Form beta.

FIG. 6 illustrates a powder X-ray diffraction pattern for imatinib mesylate Form beta.

### DETAILED DESCRIPTION OF THE INVENTION

The present invention is directed to a process for preparing crystalline form β by suspending the starting material in an appropriate solvent. The advantage of such process is that Imatinib mesylate is insoluble in the solvents of the present invention, thus, the process doesn't suffer from loss of the product in the mother liquor of the crystallization, hence, providing crystalline form β in high yields. Also, since this transformation requires only a small amount of solvent, it is beneficial for large scale operations.

As used herein powder X-ray diffraction (PXRD) patterns refer to those PXRD's obtained using copper radiation at a wavelength of λ=1.541 Å. Where indicated relative intensities reflect the intensity of one peak intensity compared to the highest peak intensity (which is set at 100) in the PXRD pattern and reflects the relative intensities for one measured PXRD.

As used herein, the term “crystalline imatinib mesylate form β” refers to crystalline imatinib mesylate which may be characterized by a powder XRD pattern having at least five peaks selected from the list consisting of peaks at about 9.7, 13.9, 14.7, 17.5, 18.2, 20.0, 20.6, 21.1, 22.1, 22.7, 23.8, 29.7 and 30.8±0.2 degrees two theta.

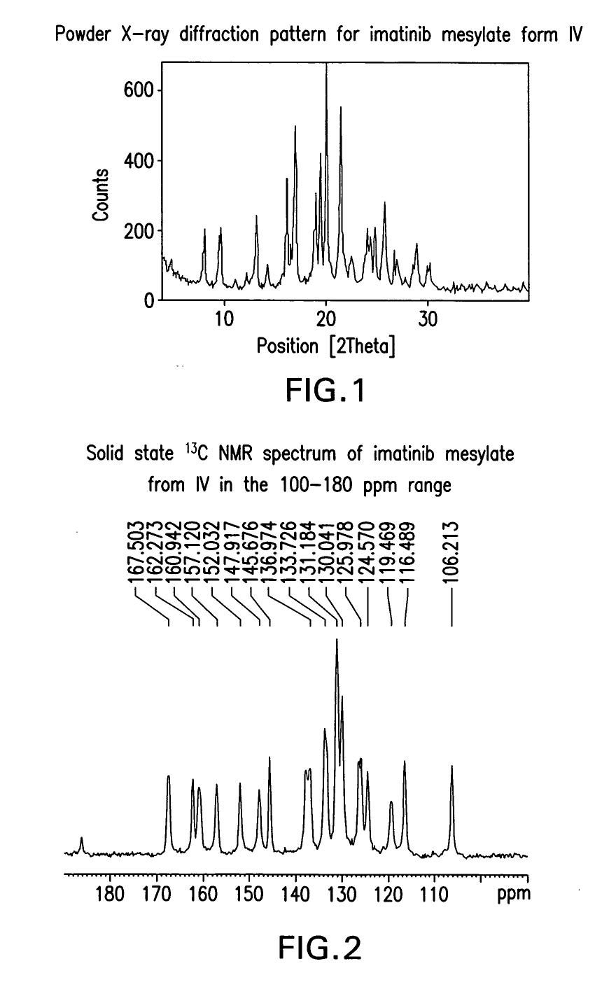

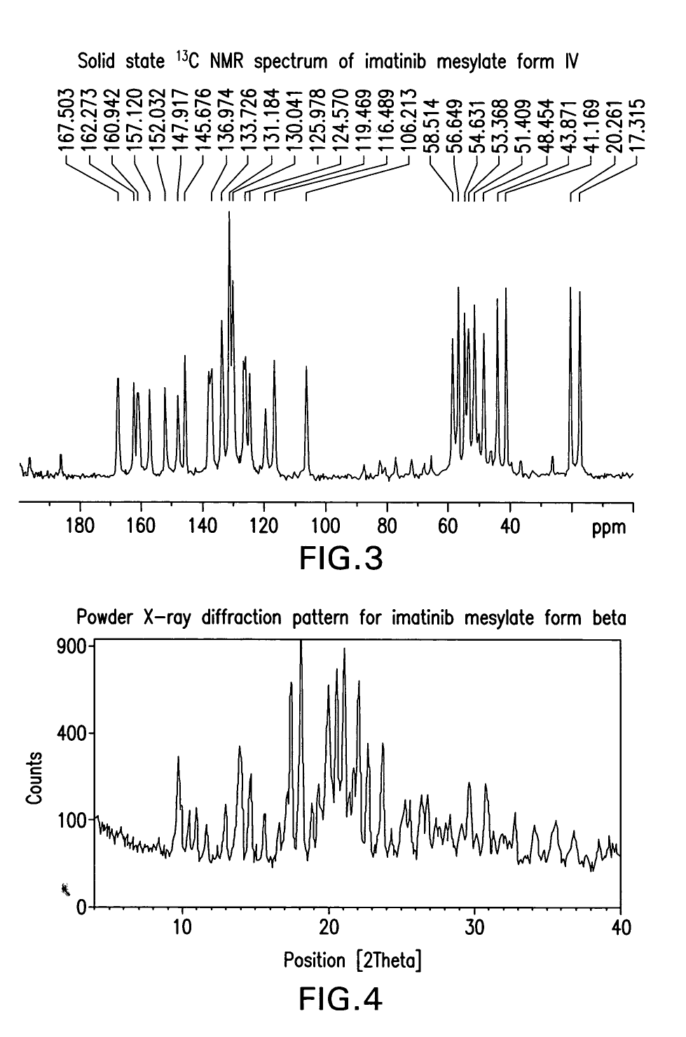

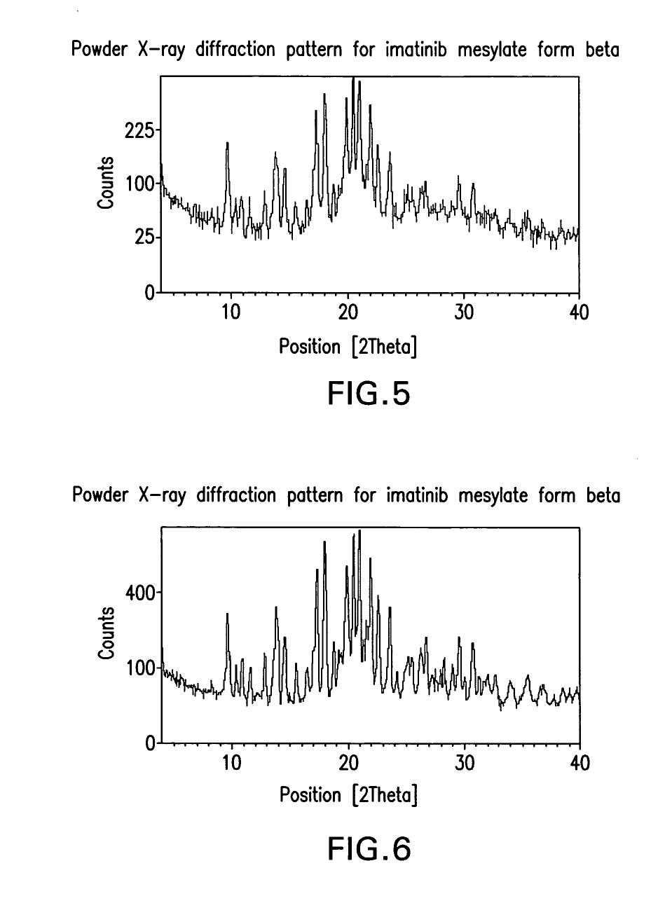

As used herein, the term “crystalline imatinib mesylate form IV” refers to crystalline imatinib mesylate characterized by data selected from the group consisting of: at least one of a powder XRD pattern with peaks at about 8.1, 9.7, 17.0, 20.1, and 21.5±0.2 degrees two-theta; a powder XRD pattern with peaks at about 8.1, 9.7, 13.2, 16.2, and 17.0±0.2 degrees two-theta; a powder XRD pattern having peaks at about 8.1, 9.7, 16.2, 17.0 and 21.5±0.2 degrees two-theta; a PXRD pattern having at least five peaks selected from the list consisting of peaks at about 8.1, 9.7, 13.2, 14.3, 16.2, 17.0, 24.1, 24.8, 25.8, 26.6, 28.9, 30.3±0.2 degrees two-theta; a powder XRD pattern depicted in FIG. 1; a solid-state ¹³C NMR spectrum with signals at about 162.3, 160.9, 157.1±0.2 ppm; a solid-state ¹³C NMR spectrum having chemical shifts differences between the signal exhibiting the lowest chemical shift and another in the chemical shift range of 100 to 180 ppm of about 56.1, 54.7 and 50.9±0.1 ppm, a ¹³C NMR spectrum depicted in FIG. 2, and a and a solid-state ¹³C NMR spectrum depicted in FIG. 3; wherein the signal exhibiting the lowest chemical shift in the chemical shift area of 100 to 180 ppm is typically at about 106.2±1 ppm. Crystalline imatinib mesylate Form IV is an ethanol solvate.

Crystalline Imatinib mesylate form IV can be provided, for example, by the process disclosed in co-pending International Patent Application No. PCT/US2007/010321 filed Apr. 27, 2007 (published as WO 2007/136510), incorporated herein by reference. The process described therein to prepare form IV comprises providing a solution of imatinib mesylate comprising imatinib mesylate and ethanol; cooling to a temperature of about 10° C. to about −50° C. to obtain a precipitate of said crystalline form; and recovering the said crystalline form.

The imatinib mesylate solution may be prepared by combining imatinib base, ethanol and methanesulfonic acid. Preferably, this process comprises: suspending imatinib base in ethanol at a temperature below 0° C.; admixing methanesulfonic acid in stoichiometric amount; and maintaining the mixture at below 0° C. to obtain a solution of imatinib mesylate. Preferably, the imatinib base is suspended in ethanol at a temperature of about 0° C. to about −40° C., more preferably, at about −10° C. Preferably, the said mixture is maintained at a temperature of about 0° C. to about −20° C., preferably about −5° C. Preferably, maintaining is by continuous stirring. It is worthy to note that in this case, the solution may be short lived and crystallization occurs shortly thereafter.

The present invention provides a process for preparing crystalline form β comprising suspending crystalline Imatinib mesylate form IV in a solvent selected from the group consisting of: a ketone, a cyclic ether, an ester or mixtures thereof.

Preferably, the ketone is a C₃-C₆ ketone, more preferably, acetone, methyl ethyl ketone, isobutyl methyl ketone, or mixtures thereof. Preferably, the cyclic ether is a C₃-C₅ cyclic ether, more preferably, tetrahydrofuran, methyltetrahydrofuran, dioxolane, or mixtures thereof. Preferably, the ester is a C₃-C₈ ester, more preferably, ethyl acetate.

The suspension is then maintained to obtain Imatinib mesylate form β.

In one embodiment the solvent is ethylacetate. In one embodiment the solvent is tetrahydrofuran. In one embodiment the solvent is acetone.

The ratio of Imatinib mesylate form IV to the solvent is preferably about 1 to about 10 or less, preferably about 0.5 to about 10 or less, more preferably about 0.4 to about 10 (weight of Imatinib mesylate in g to volume of solvent in ml) or less. Combining crystalline Imatinib mesylate form IV with the solvent can be carried out at a temperature of about 0° C. to about 55° C., preferably at about 20° C. to about 45° C., more preferably at about 25° C. to about 35° C., even more preferably at about 30° C.

The suspension, which is a heterogeneous mixture, can be maintained for a sufficient period of time to allow the transformation of form IV into form β to occur. Preferably, the suspension is maintained at the above described temperature. Preferably, the suspension is maintained for about 1 hour to about 36 hours, more preferably for about 1 hours to about 15 hours, most preferably, for about 2 hours to about 12 hours.

The process for preparing form β can further comprise a recovery process. The recovery can be done for example, by cooling the heterogeneous mixture, isolating the obtained crystalline form β, washing the isolated crystalline and drying. The crystalline imatinib mesylate form β may be isolated and dried. Isolation can be carried out by filtration. To accelerate the filtration, the filtration can be carried out under vacuum (pressure of less than 100 mmHg). The isolated crystalline imatinib mesylate form β can then be dried. Drying can be carried out at a temperature of about 25° C. to about 65° C., such as about 50° C. A pressure of less than one atmosphere, such as vacuum (pressure of less than 100 mmHg), can be used to accelerate the drying process.

The process of preparing crystalline imatinib mesylate form β provides a crystalline imatinib mesylate form β with a yield of at least about 85% (percent of weight of imatinib mesylate form IV), preferably of at least about 90%, more preferably of 94% or more.

Having described the invention with reference to certain preferred embodiments, other embodiments will become apparent to one skilled in the art from consideration of the specification. The disclosures of the references referred to in this patent application are incorporated herein by reference. The invention is further defined by reference to the following examples describing in detail the process of the invention. It will be apparent to those skilled in the art that many modifications, both to materials and methods, may be practiced without departing from the scope of the invention.

### EXAMPLES

### XRD

XRD diffraction was performed on X-Ray powder diffractometer: Philips X'pert Pro powder diffractometer, Cu-tube, scanning parameters: CuKα radiation, λ=1.5418 A. Continuous scan at a rate of: 6 deg./min. Zero background sample holders.

### 13C NMR

The CP/MAS 13C NMR measurements were made at Bruker Avance 500 NMR US/WB spectrometer in 4-mm ZrO2 rotor. Magic angle spinning (MAS) speed was 10 kHz. As used herein, the term “13C NMR chemical shifts” refers to the shifts measured under above specified conditions, however, these shifts can slightly differ instrument to instrument and can be shifted either upfield or downfield due to the different instrumental setup and calibration used. Nevertheless the sequence of individual peaks remains identical.

### Example 1

Imatinib mesylate form IV (400 mg) was added to tetrahydrofuran (10 ml) and the suspension was stirred 2 h at 30° C. Then the suspension was cooled to 15° C., imatinib mesylate form β was recovered by filtration, washed with petrolether and dried at 50° C., 1 mBar. Yield 330 mg (89%).

### Example 2

Imatinib mesylate form IV (400 mg) was added to ethylacetate (10 ml) and the suspension was stirred 12 h at 30° C. Then the suspension was cooled to 15° C., imatinib mesylate form β was recovered by filtration, washed with petrolether and dried at 50° C., 1 mBar. Yield 350 mg (94%).

### Example 3

Imatinib mesylate form IV (400 mg) was added to acetone (10 ml) and the suspension was stirred 12 h at 30° C. Then the suspension was cooled to 15° C., imatinib mesylate form β was recovered by filtration, washed with petrolether and dried at 50° C., 1 mBar. Yield 350 mg (94%).

### Example 4

### Preparation of Imatinib Mesylate Form IV

Imatinib base (3 g) was suspended in ethanol (60 ml, 96%) at −10° C. Methanesulfonic acid (0.375 ml) was added with stirring and the suspension was stirred for additional 20 min at −5° C. obtaining thus the solution of imatinib mesylate. Than the solution was allowed to crystallize without stirring at −5° C. for 3 hours. t-Butyl methyl ether (50 ml) was added, the white solid was filtered, washed with petrolether (50 ml) and dried in a stream of nitrogen for 1 h to obtain imatinib mesylate Form IV (3.18 g, yield: 89%).

What is claimed is:

1. A method of preparing crystalline Imatinib mesylate form β comprising: suspending crystalline imatinib mesylate form IV in a solvent selected from the group consisting of a ketone, a cyclic ether, an ester or mixtures thereof forming a suspension comprising crystalline imatinib mesylate form β.

2. The method of claim 1, wherein the ratio of crystalline imatinib mesylate form IV to solvent is less than about 1 g of crystalline imatinib mesylate form IV to about 10 ml of solvent.

3. The method of claim 1, wherein the suspending is carried out at a temperature of about 0° C. to about 55° C.

4. The method of claim 1, wherein the suspension is maintained for a period of about 1 hour to about 36 hours.

5. The method of claim 1, further comprising recovering the crystalline Form β.

6. The method of claim 1, wherein the solvent is a C₃-C₈ ester.

7. The method of claim 6, wherein the solvent is ethylacetate.

8. The method of claim 1, wherein the solvent is a C₃-C₅ cyclic ether.

9. The method of claim 8, wherein the solvent is tetrahydrofuran.

10. The method of claim 8, wherein the solvent is methyltetrahydrofuran.

11. The method of claim 8, wherein the solvent is dioxolane.

12. The method of claim 1, wherein the solvent C₃-C₆ ketone.

13. The method of claim 12, wherein the solvent is acetone.

14. The method of claim 12, wherein the solvent is methyl ethyl ketone.

15. The method of claim 12, wherein the solvent is isobutyl methyl ketone.
